(19) United States
(12) Patent Application Publication
(10) Pub. No.: US 2014/0107694 A1
(43) Pub. Date: Apr. 17, 2014

(54) INFERIOR VENA CAVA FILTER

(71) Applicant: Daniel Sheng Wang, Moreland Hills, OH (US)

(72) Inventor: Daniel Sheng Wang, Moreland Hills, OH (US)

(21) Appl. No.: 14/052,276

(22) Filed: Oct. 11, 2013

Related U.S. Application Data
(60) Provisional application No. 61/712,562, filed on Oct. 11, 2012.

(51) Int. Cl. A61F 2/01

(52) U.S. Cl. CPC A61F 2/01
USPC 606/200

(57) ABSTRACT

An inferior Vena Cava (“IVC”) filter is formed of two adjacent structures such as polyhedral, each having a number of sides positioned to avoid side-wise contact with the caval wall. The structures form a first filter basket and a second filter basket respectively, with the filter baskets joined by a center structure around a center axis of the IVC filter. In this configuration, the IVC filter advantageously self-aligns within the IVC, while permitting bidirectional or directionally independent deployment, use, and retrieval. While polyhedra may usefully be configured with no sides parallel to the IVC wall to mitigate attachment to the IVC wall, the structures of the filter baskets may also or instead use curved members distal to the center axis that contact the IVC wall only at a single tangent point or area.

### CROSS-REFERENCE TO RELATED APPLICATIONS

This application claims the benefit of U.S. Provisional Application No. 61/712,562 filed on Oct. 11, 2012, the entire content of which is hereby incorporated by reference.

### BACKGROUND

1. Field

The invention generally relates to inferior vena cava filters.

2. Description of the Related Art

Inferior Vena Cava (IVC) filters prevent pulmonary embolism by catching blood clots in the vascular system before they reach the lung. IVC filters are used in patients with high risk of pulmonary embolism or when anticoagulation therapy (blood thinner medications) cannot be used. Originally, such filters were placed into human bodies as permanent devices. However, increasing evidence indicated that long term-use leads to complications such as caval occlusion resulting in bilateral severe leg swelling, as well as filter erosion into the caval wall or adjacent organs.

Retrievable IVC filters have been increasingly preferred over permanent filters during the past decade to avoid potential risks of long-term complications from permanent filters. While current designs address the retrievability of IVC filters, these new filters also introduce other potential problems. In particular, current filters generally have extending struts (arms or legs) to secure to the caval wall. Therefore, penetration of the struts beyond the caval wall is unavoidable. If significant, penetration can cause retropetoneal bleeding, adjacent organ injury and abdominal pain. In some occasions, the struts fracture, with the resulting fragments migrating downstream in the blood flow. These migrated fracture fragments can cause fatal arrhythmia and rupture of the heart chamber. Furthermore, the majority of current filters do not have a self-centralization mechanism due to a conical shaped design. These filters often tilt once inserted into the human body. A significantly tilted filter may cause difficultly or failure in retrieval. Less common retrievable filters use polyhedral structures. While providing greater structural integrity, these structures generally present increased contact surface to the caval wall, resulting in a limited implant window (2 weeks) before the filter embeds itself to the vessel wall in a manner that renders retrieval difficult or impossible.

Various existing IVC filters are described, by way of example, in U.S. Pat. No. 8,105,349; U.S. Pat. No. 8,092,485; U.S. Pat. No. 7,704,267; and U.S. Pat. Pub. No. 2009/0299403. The disclosures of these patents and publications are hereby incorporated by reference in their entirety.

There remains a need for an improved, retrievable IVC filter.

### SUMMARY

An inferior Vena Cava (“IVC”) filter is formed of two adjacent structures such as polyhedral, each having a number of sides positioned to avoid side-wise contact with the caval wall. The structures form a first filter basket and a second filter basket respectively, with the filter baskets joined by a center structure around a center axis of the IVC filter. In this configuration, the IVC filter advantageously self-aligns within the IVC, while permitting bidirectional or directionally independent deployment, use, and retrieval. While polyhedra may usefully be configured with no sides parallel to the IVC wall to mitigate attachment to the IVC wall, the structures of the filter baskets may also or instead use curved members distal to the center axis that contact the IVC wall only at a single tangent point or area.

An IVC filter may be formed of a number of sides joined at a number of vertices in a deployed form including a center structure having an axis. The IVC filter may include a first filter basket including a first number of sides each coupled at a first number of vertices to form a first polyhedron. A first one of the first number of vertices may be coupled to the center structure and three or more of the vertices are positioned about the axis. The IVC filter further may include a second filter basket including a second number of sides each coupled at a second number of vertices to form a second polyhedron. A first one of the second number of vertices may be coupled to the center structure and three or more of the vertices may be positioned about the axis. The IVC filter may be configured so that none of the sides on a convex hull of the device are parallel to the axis, or more generally such that no sides of the convex hull would rest along the wall of the IVC when the filter is placed for use.

In another aspect, the filter may include a first filter basket having a first polyhedral geometry and no free ends, where the first polyhedral geometry includes a first number of contact points at a number of vertices arranged to contact an interior wall of an inferior vena cava (IVC) when the filter is placed for use in the IVC. The filter may include a second filter basket having a second polyhedral geometry and no free ends, where the second polyhedral geometry includes a second number of contact points at a number of vertices arranged to contact the interior wall of when the IVC filter is placed for use in the IVC. The filter may also include a narrow center joining the first filter basket and the second filter basket into a self-centering, bi-directional, IVC filtering structure.

In another aspect an IVC filter may include a first filter basket having at least one cross section shaped and sized to fit within and engage a wall of an inferior vena cava at a number of discrete locations, and a second filter basket having at least one cross section shaped and sized to fit within and engage the wall of the inferior vena cava at a second number of discrete locations. The IVC filter may include a center structure joining the first filter basket and the second filter basket, where the center structure has a cross section with a diameter smaller than the cross section of the first filter basket and the cross section of the second filter basket.

The filter baskets may include a number of sides coupled at vertices to form polyhedrons. The filter baskets may be joined to the center structure with none of the sides on a convex hull of the filter baskets parallel to the axis of the IVC filter.

In a further aspect, none of the sides of the filter baskets lie on a perimeter of an axial cross section of the device. The center structure may join one of the vertices of one filter basket to the vertices of the other filter basket. The filter baskets may be formed of surgical stainless steel. The sides of the filter baskets may be formed of a number of lengths of stainless steel wire with no free ends. In some aspects, the lengths of stainless steel wire do not terminate at the center structure. All of the number of lengths may terminate at a first retrieval fixture coupled to the first filter basket or a second retrieval fixture coupled to the second retrieval basket.

The filter baskets may be formed of a shape memory alloy. The shape memory alloy may include nickel titanium. The filter baskets may also or instead be formed of an MRI-compatible material. The IVC filter may be shaped and sized to self-align within the IVC. The IVC filter may be a bi-directional filter having two opposing functional alignments. The filter baskets may form two sequential filters in a path of flow through the IVC. The filter baskets may be collapsible from the deployed form into a collapsed form to permit deployment and retrieval through a minimally invasive surgical tool and/or using an endovascular technique. The IVC filter may elastically return to the deployed form from the collapsed form in the absence of external forces. The IVC filter may be formed of a shape memory alloy that returns to the deployed form upon heating above a transition temperature.

In another aspect a filter disclosed herein includes a first filter basket with a polyhedral geometry and no free ends. The polyhedral geometry may include a number of contact points at a number of vertices arranged to contact an interior wall of an inferior vena cava when the filter is placed for use in the IVC. The filter may include a second filter basket with a polyhedral geometry and no free ends. As with the first filter basket, the polyhedral geometry of the second basket may include a number of contact points at a number of vertices arranged to contact an interior wall of the IVC. The first and second filter baskets may be joined at a narrow center. This configuration creates a self-centering, bi-directional, IVC filtering structure.

One or more of the contact points of the filter may include a spike to secure the filter to the interior wall of the IVC. The spike may be about two and a half millimeters long, between two and three millimeters long, or between one and four millimeters long. The first filter basket and second filter basket may be collapsible to facilitate deployment retrieval through a minimally invasive endovascular tool. One or more of the contact points may be formed by a vertex having a wide angle, such as an angle greater than ninety degrees.

The filter may include a first retrieval fixture on the first filter basket. The first retrieval fixture may include a cylinder with a narrowed side wall configured to removably and replaceably receive a snare. The filter may include a second retrieval fixture on the second filter basket. The first filter basket and second filter basket may be formed of by a number of wires, and the first retrieval fixture and the second retrieval fixture may secure a number of terminal ends of the number of wires.

The filter may be configured so that none of the sides of the first filter basket and second filter basket contact the interior wall of the IVC when the filter is placed for use in the IVC. The filter may also or instead be configured so that no sides of the first filter basket and the second filter basket are in parallel contact with the interior wall of the IVC when the filter is placed for use in the IVC. The first filter basket and the second filter basket may form filtering structures to catch clots within blood flowing through the IVC when the filter is placed for use.

### BRIEF DESCRIPTION OF THE FIGURES

The invention and the following detailed description of certain embodiments thereof may be understood by reference to the following figures, wherein similar reference characters denote similar elements throughout the several views.

FIG. 1A shows a side view of an IVC filter.

FIG. 1B shows a top view of an IVC filter.

FIG. 1C shows a perspective view of an IVC filter.

FIG. 1D shows a perspective view of an IVC filter.

FIG. 2 shows a side view of a collapsed IVC filter.

FIG. 3A shows a side view of an IVC filter.

FIG. 3B shows a top view of an IVC filter.

FIG. 3C shows a perspective view of an IVC filter.

FIG. 3D shows a perspective view of an IVC filter.

FIG. 4A shows a side view of an IVC filter.

FIG. 4B shows a top view of an IVC filter.

FIG. 4C shows a perspective view of an IVC filter.

FIG. 4D shows a perspective view of an IVC filter.

FIG. 5A shows a side view of an IVC filter.

FIG. 5B shows a top view of an IVC filter.

FIG. 5C shows a perspective view of an IVC filter.

FIG. 5D shows a perspective view of an IVC filter.

FIG. 6A shows a side view of an IVC filter.

FIG. 6B shows a top view of an IVC filter.

FIG. 6C shows a perspective view of an IVC filter.

FIG. 6D shows a perspective view of an IVC filter.

FIG. 7A shows a side view of an IVC filter.

FIG. 7B shows a top view of an IVC filter.

FIG. 7C shows a perspective view of an IVC filter.

FIG. 7D shows a perspective view of an IVC filter.

### DETAILED DESCRIPTION

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Ranges of values and/or numeric values are provided herein as examples only, and do not constitute a limitation on the scope of the embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, like reference characters designate like or corresponding parts throughout the figures. Additionally, in the following description, it is understood that terms such as “first,” “second,” “center,” “top,” “bottom” and the like, are words of convenience and are not to be construed as limiting terms.

One skilled in the art will recognize that the embodiments shown in the figures are provided as examples only, and deviations from these embodiments are still within the scope of this disclosure. Similarly, any sizes or shapes described herein are provided as examples only, and deviations from these embodiments are still within the scope of this disclosure.

Certain embodiments of interior vena cava filters are now be described more fully hereinafter with reference to the accompanying figures. It will be understood that the principles of the invention are not so limited, and may be usefully employed in any context where a combination of structural rigidity, self-centering, bidirectional operation, and low contact surfaces might be advantageous, such as other blood or fluid filtering applications. As such, the illustrated embodiments are provided by way of example only, and not to limit the scope of the invention.

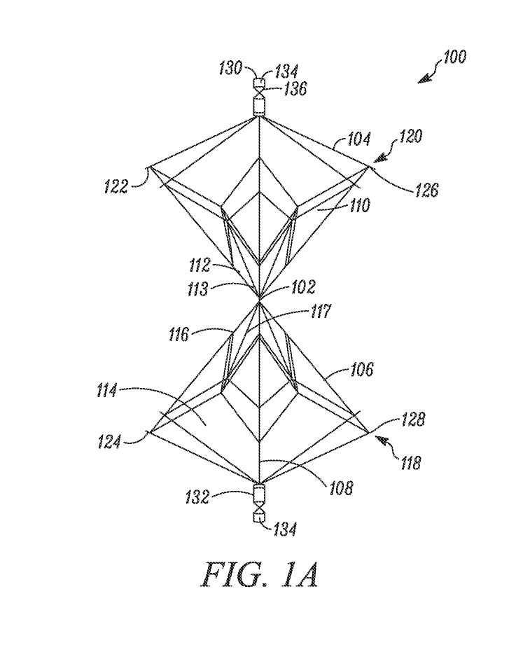

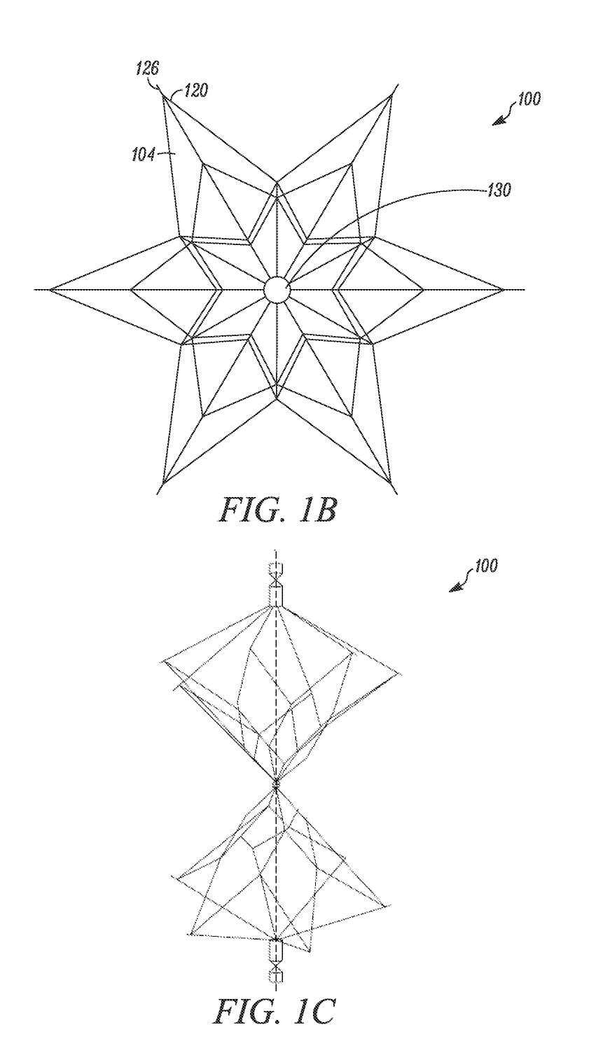

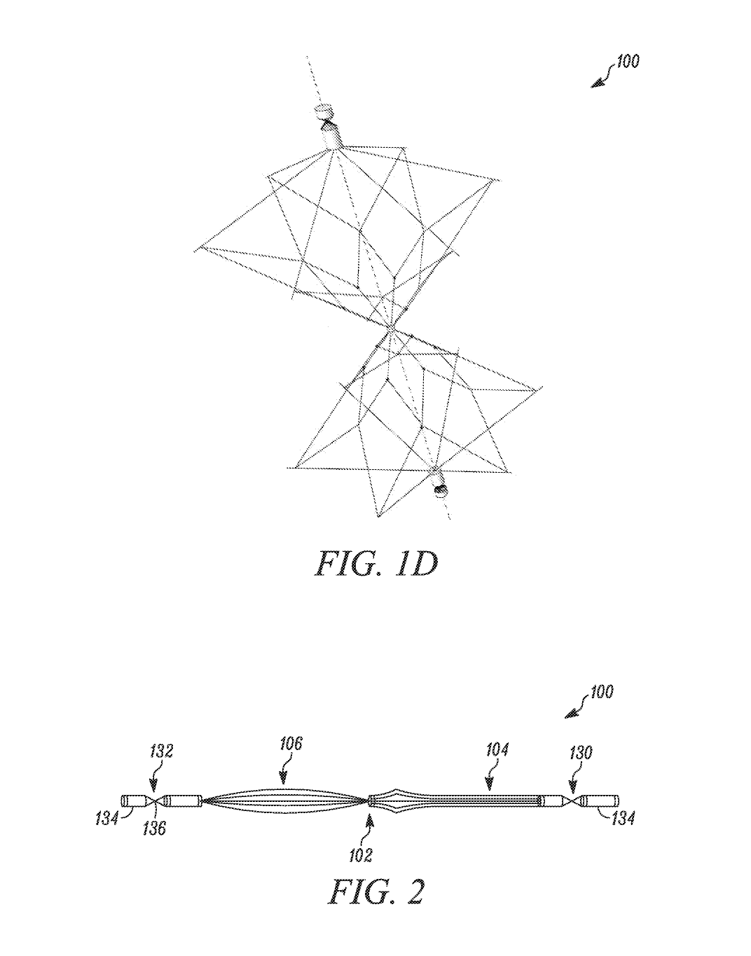

FIG. 1A shows a side view of an IVC filter. The filter 100 may be formed of a center structure 102, a first basket 104, and a second basket 106. The center structure 102 creates a narrow section of the filter 100 between the first basket 104 and the second basket 106, and defines an axis 108 for the filter 100 about which the center structure 102 and the filter baskets 104, 106 are formed. The first filter basket 104 may include a number of sides 110 and a corresponding number of vertices 112. The sides 110 and the vertices 112 may form a polyhedron. One of the vertices 112 may be coupled to the center structure 102 and to three or more of the other vertices 112 positioned about the axis 108. The second filter baskets 106 may include a number of sides 114 and a corresponding number of vertices 116. The sides 114 and the vertices 116 may be arranged such that they also form a polyhedron. The sides 110, 114 may generally lie on a perimeter of an axial cross section of the filter 100. One or more of the vertices 112, 116 may be a wide angle vertex, e.g., with an angle greater than ninety degrees, in order to mitigate possible piercing or rending of the interior vena cava wall. In some filters, all of the vertices 112, 116 may be formed by a wide angle.

One of the vertices 116 may be coupled to the center structure 102 and to three or more of the vertices 116 positioned about the axis 108. The center structure 102 may join the first filter basket 104 to the second filter baskets 106. As shown, the vertices 112 of the first basket 104 are joined to the center structure 102 by lengths 113 and the vertices 116 are joined to the center structure 102 by lengths 117. None of the sides 110, 114 on either the first or second filter basket 104, 106, which forms a convex hull, are parallel to axis 108. Since the sides are not parallel to the axis 108, the sides 110, 114 are not in contact with the interior wall of the IVC when the filter is placed for use in the IVC, which may advantageously mitigate attachment to the IVC wall during prolonged use of the filter. When deployed in the IVC the filter baskets 104, 106 form filtering structures that are used to catch blood clots within the blood flow through the IVC. The combination of the two filter baskets 104, 106 creates a substantially hourglass shaped filter 100.

The use of two basket filters 104, 106 as described above provides numerous advantages. The dual baskets allow the IVC filter 100 to stabilize and self-align around the center structure 102. This is due to the two areas of contact 118, 120 formed about the two filter baskets 104, 106. The two-basket design also allows for bidirectional deployment in the IVC from either the jugular end or the femoral end. Further, the shape minimizes the surface area of contact with the blood vessel wall, thus limiting or mitigating adhesion to the vessel over time. In contrast, a cylindrical deployment or other deployment with large contact surfaces will tend to embed in the wall. The two-basket design also allows for the deployment of two sequential filters in the flow path of the IVC. Further, the two-basket design allows the surgeon to retrieve the filter 100 from either the jugular end or the femoral end of the filter, or more generally bi-directionally. At the same time, the use of closed structures such as polyhedral or the like provides structural stability to the filter, thus preventing collapse, shape distortion, breakage or free ends, or other unwanted artifacts of other structural designs. The filter 100 may in general be deployed and retrieved using minimally invasive surgical tools and/or endovascular techniques.

The contact areas 118, 120 may include one or more spikes 122, 124, 126, 128 to secure the filter 100 to the interior wall of the IVC. The spikes 122, 124, 126, 128 may be about one and one half millimeters long, two and one half millimeters long, between two and three millimeters long, between one and five millimeters long, or any other suitable length consistent with the deployment of a retrievable IVC filter as contemplated herein. The spikes 122, 124, 126, 128 may engage the wall of the vessel once the IVC filter 100 is deployed within the IVC.

FIG. 1B shows a top view of an IVC filter 100. The IVC filter is shown from the end with the first basket 104 obscuring the second basket 106. The contact areas 120 may include spikes 126. The retrieval fixture 130 is located on the axis that is substantially in the center of the IVC filter 100.

FIGS. 1C and 1D show perspective views of an IVC filter 100.

FIG. 2 shows a side view of a collapsed IVC filter. The filter 100 may, for example, be the filter 100 described above or any of the other filters described herein. The collapsed state provides a narrow cross section suitable for passing through a minimally invasive surgical tool or a relatively small diameter body opening or passage. This configuration permits deployment and retrieval of the filter 100 using an endovascular technique. The filter baskets described herein may be formed of surgical stainless steel, a shape memory alloy such as nickel titanium or the like, an MRI-compatible material (e.g., any material or combination of materials that does not substantially interfere with MRI imaging or the operation of MRI imaging systems), or any other appropriate material. In some filters, the material forming the filter baskets may be formed such that the material lengths do not have any free ends. By securing materials on two ends, this advantageously reduces the chances for portions of the device to break off and separate from the device. In some filters, the material forming the filter baskets 104, 106 may have ends that terminate in the center structure 102. For example, the filter baskets 104, 106 may be formed of a number of lengths of surgical stainless steel wire. By terminating these lengths of wire at the center structure 102 and/or at the terminal retrieval fixtures 130, 132, the filter baskets 104, 106 can be constructed with no ends of the forming material disposed within the polyhedral structures thereof where the ends might become disconnected. Similarly, some or all of the lengths terminate at the first retrieval fixture 130 or at the second retrieval fixture 132 (or some combination of these), and pass through the center structure 102 without terminating in the center structure 102. The use of shape memory alloys allows the filter 100 to return to an original shape through a variety of means, which include exposing the filter 100 to external forces or heating the filter 100 to a temperature above its transition temperature. It will be appreciated that while a nickel-titanium alloy (commonly referred to as nitinol) is one generally available and suitable shape memory alloy, a wide variety of shape memory alloys and other shape memory materials are known, including materials that respond to temperature, electrical fields, and other stimuli to return to a “remembered” shape, many of which may be suitably employed as the shape memory material contemplated herein.

The retrieval fixture 130 may include a cylinder 134 and a narrowed sidewall 136 or other indentation. The narrowed sidewall 136 is configured to removably and replaceably receive a snare, such as by providing a portion of the cylinder that, once snared, will securely retain the snare on the retrieval fixture 130. A snare may include a string, suture loop, or the like that, when positioned around the retrieval fixture 130 and pulled tight, closes around the narrowed side wall 136 and allows the filter 100 to be pulled into a sheath and then collapsed for retrieval, or otherwise permits an application of force to position the filter 100 as desired. The narrowed sidewall 136 may include a gib, hook, latch, pin, spike, wedge, or the like to facilitate manipulation of the filter 100. The retrieval fixture 130 may be on either end of the filter 100 or on both ends. Further, the filter 100 may be removed from the IVC from the jugular end or the femoral end.

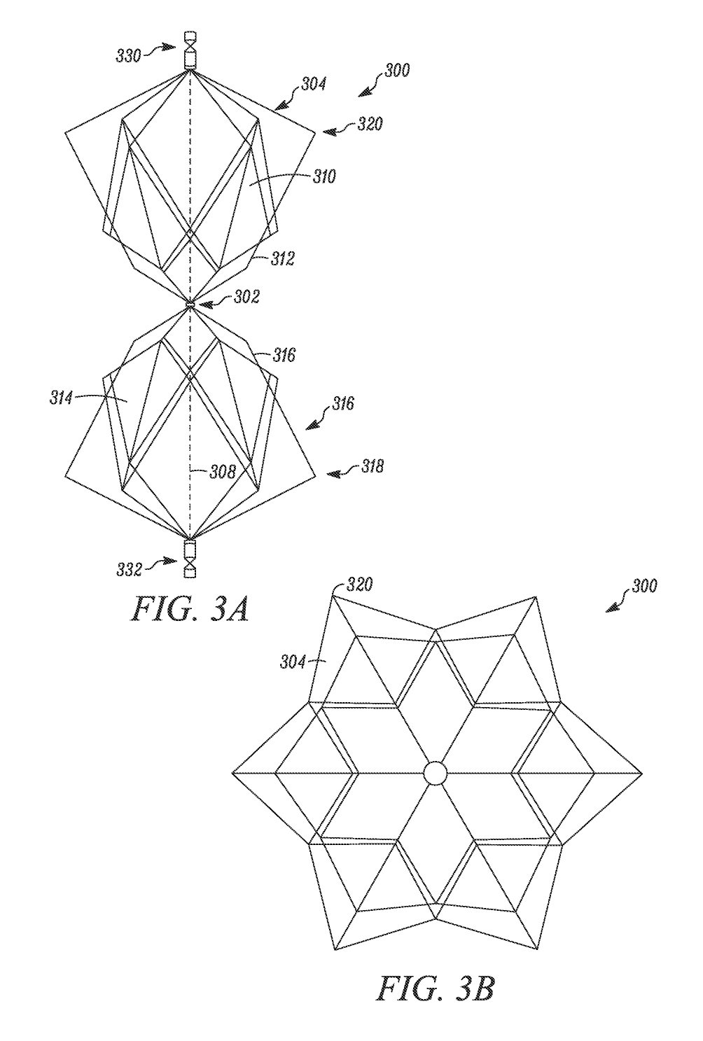

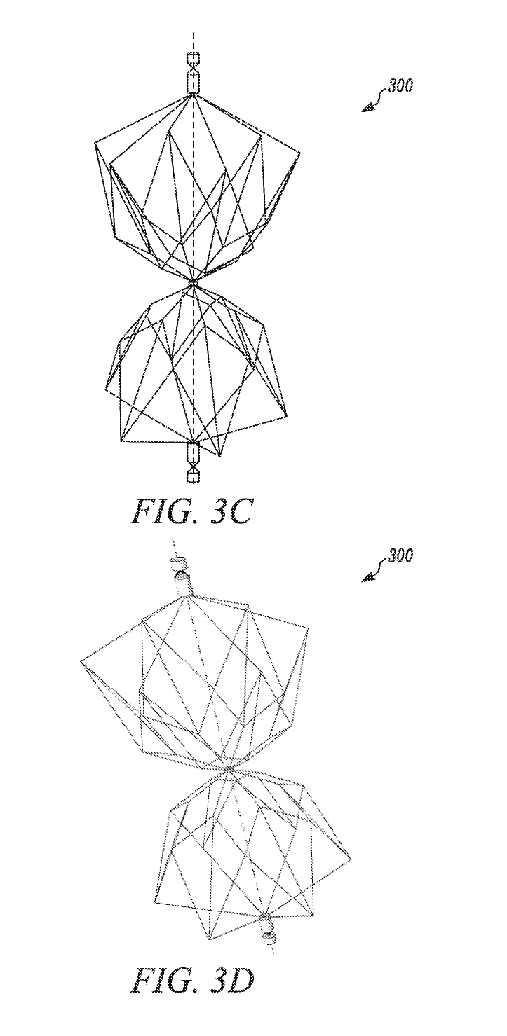

It will be appreciated that the disclosed polyhedral structures may take shapes other than the ones depicted in the figures included herein. Nevertheless, additional embodiments will now be described as depicted in FIGS. 3A-7D, where the IVC filters include two substantially adjacent structures coupled by a narrow center.

FIG. 3A shows a side view of an IVC filter. Similar to the embodiment shown in FIG. 1A, the filter 300 may include a center structure 302, a first basket 304, and a second basket 306. The center structure 302 may define an axis 308 about which the center structure 302 is constructed, and may create a narrow section of the filter 300. The first filter basket 304 may include a number of sides 310 and a corresponding number of vertices 312. The sides 310 and the vertices 312 may be assembled such that they form a polyhedron. One of the vertices 312 may be coupled to the center structure 302 and further coupled to three or more of the vertices 312 positioned about the axis 308 along a number of sides 310. The second filter baskets 306 may include a number of sides 314 and a corresponding number of vertices 316. The sides 314 and the vertices 316 may be assembled such that they also form a polyhedron. The sides 310, 314 may be arranged on a perimeter of the axial cross section of the filter 300. One or more of the vertices 312, 316 may form a wide angle, e.g., an angle greater than ninety degrees, in order to mitigate possible piercing or rending of the IVC wall. The embodiment illustrated in FIG. 3A has vertices with a wider angle than the embodiment of FIG. 1A. The wider angle of the vertices 312, 316 creates less of a sharp contact area 318, 320 where the baskets contact the IVC when placed for use.

The embodiment of FIG. 3A may also include a retrieval fixture 330, 332. Additionally, although not depicted in FIG. 3A or 3B, the filter 300 may include spikes or the like to secure the filter 300 against movement when placed for use in the IVC.

FIG. 3B shows a top view of an IVC filter. The filter 300 is shown from the end with the first basket 304 obscuring the second basket 306.

FIGS. 3C and 3D show perspective views of an IVC filter 300.

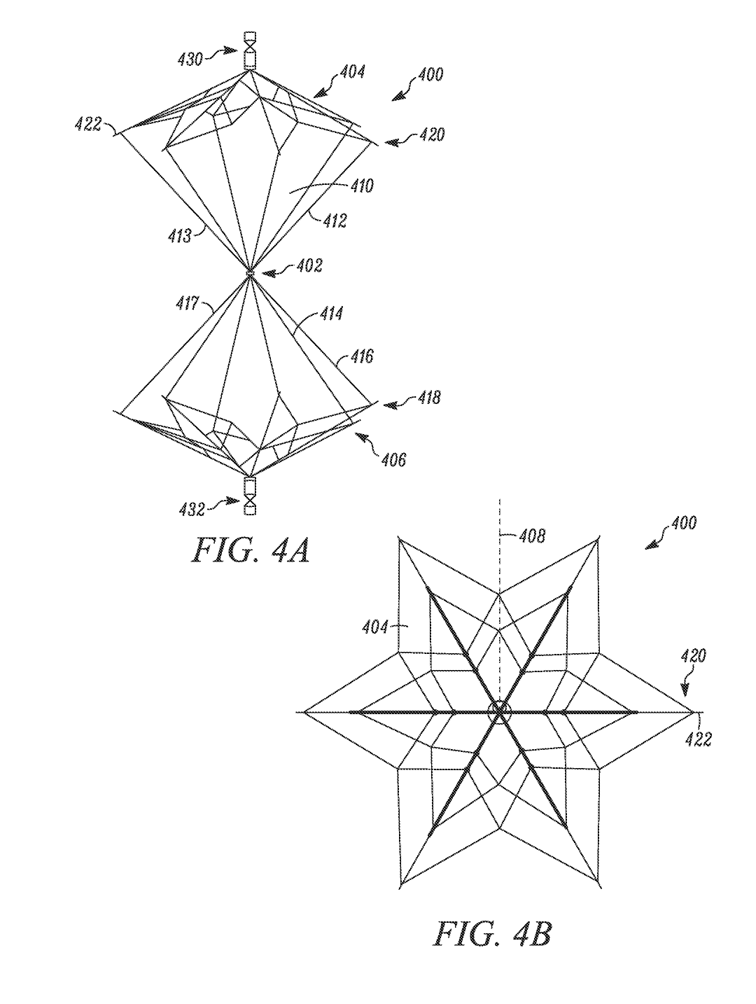

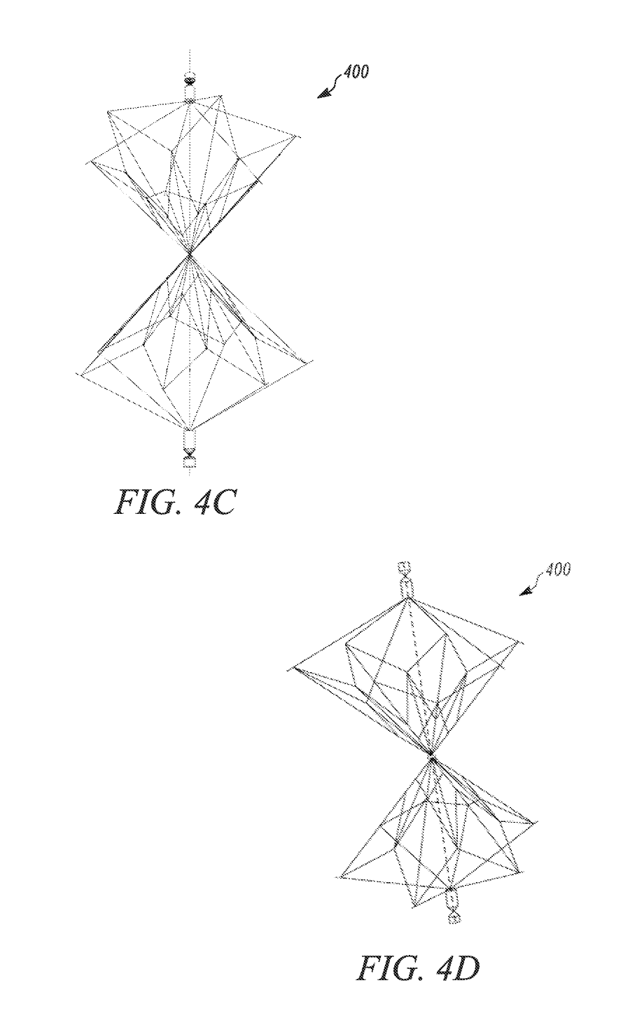

FIG. 4A shows a side view of an IVC filter. Similar to the embodiments shown in FIGS. 1A and 3A, the filter 400 may include a center structure 402, a first basket 404, and a second basket 406. The center structure 402 defines an axis 408 about which the center structure 402 may be constructed. The center structure 402 creates a narrow section of the filter 400. The first filter baskets 404 may include a number of sides 410 and a corresponding number of vertices 412. The sides 410 and the vertices 412 may be assembled such that they form a polyhedron. One of the vertices 412 may be coupled to the center structure 402 and to three or more of the vertices 412 positioned about the axis 408. The second filter baskets 406 may include a number of sides 414 and a corresponding number of vertices 416. The sides 414 and the vertices 416 may be assembled such that they also form a polyhedron. The sides 410, 414 lie on a perimeter of the axial cross section of the filter 400. One or more of the vertices 412, 416 may form a wide angle, e.g., an angle greater than ninety degrees, in order to mitigate possible piercing or rending of the IVC wall. The embodiment of FIG. 4A is similar to the embodiment of FIG. 1A, except that the areas of contact 418, 420 are located closer to the retrieval fixtures 430, 432 in the embodiment of FIG. 4A than the areas of contact 118, 120 are in the embodiment of FIG. 1A. In other words, the lengths 413, 417 in the filter 400 are longer than the lengths 113, 118 in the filter 100 of FIG. 1A. Additionally, the filter 400 may include spikes 422 or the like to secure the filter 300 against movement when placed for use in the IVC. FIG. 4B shows a top view of an IVC filter. The filter 400 is shown from the end with the first basket 404 obscuring the second basket 406. FIGS. 4C and 4D show perspective views of an IVC filter 400.

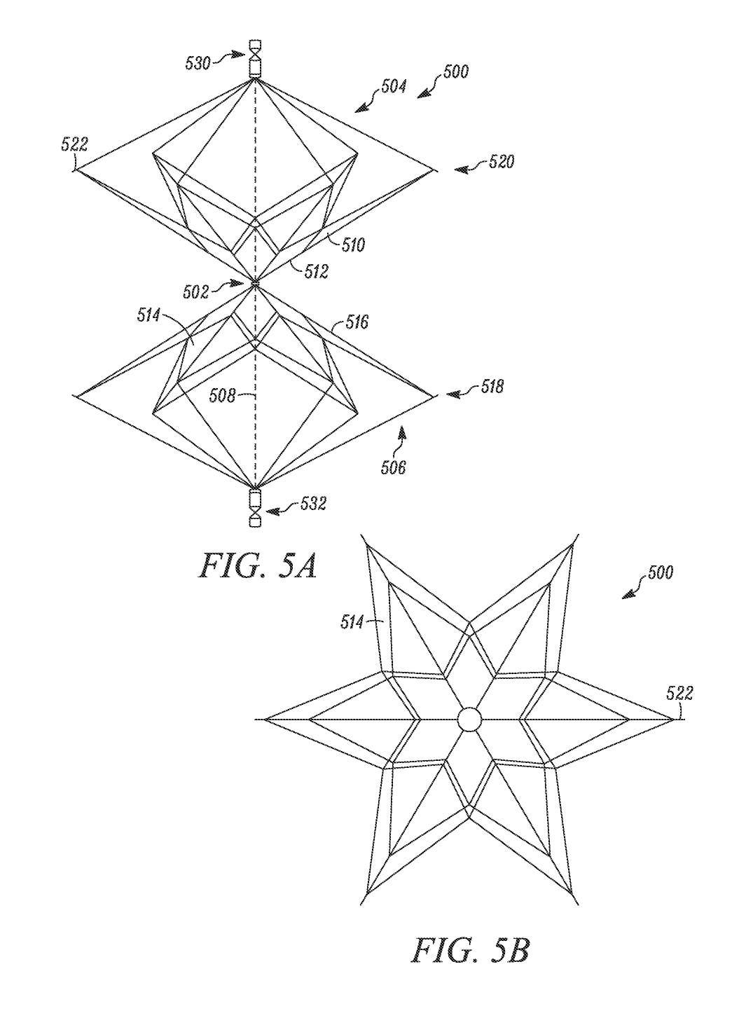

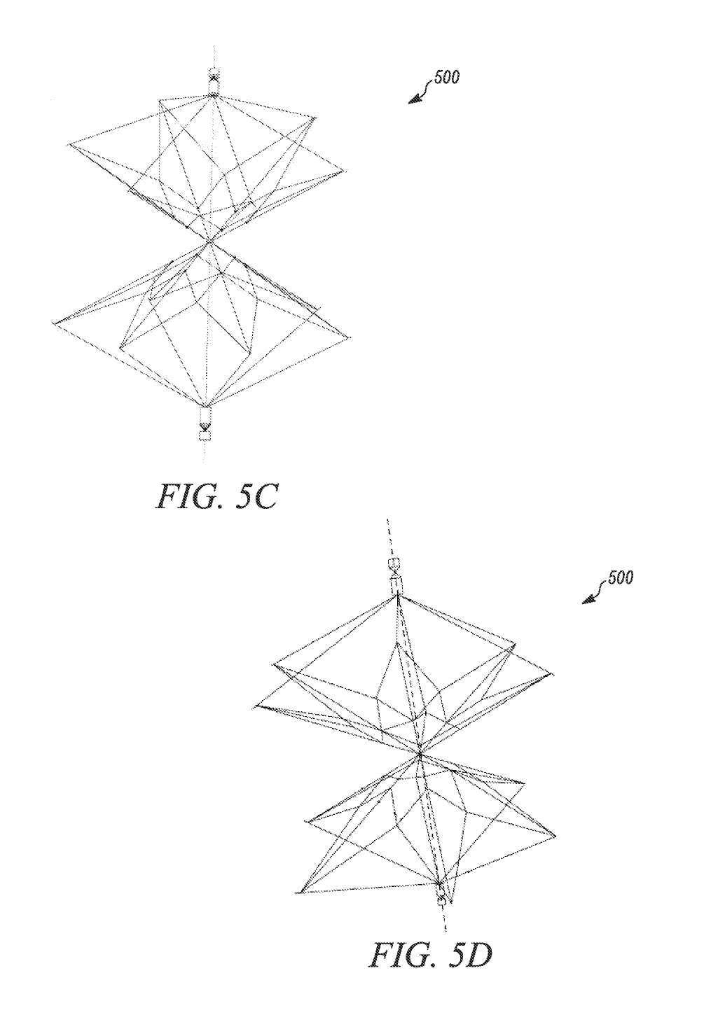

FIG. 5A shows a side view of an IVC filter. The filter 500 may include a center structure 502, a first basket 504 formed by sides 510 and vertices 512, and a second basket 506 formed by sides 514 and vertices 516. In the embodiment of FIG. 5A, the areas of contact 518, 520 are located substantially midway between the retrieval fixtures 530, 532 and the center structure 502. Additionally, the IVC filter 500 may include spikes 522 or the like to secure the filter 500 against movement when placed for use in the IVC. FIG. 5B shows a top view of an IVC filter such as the filter 500 of FIG. 5A. FIGS. 5C and 5D show perspective views of an IVC filter 500.

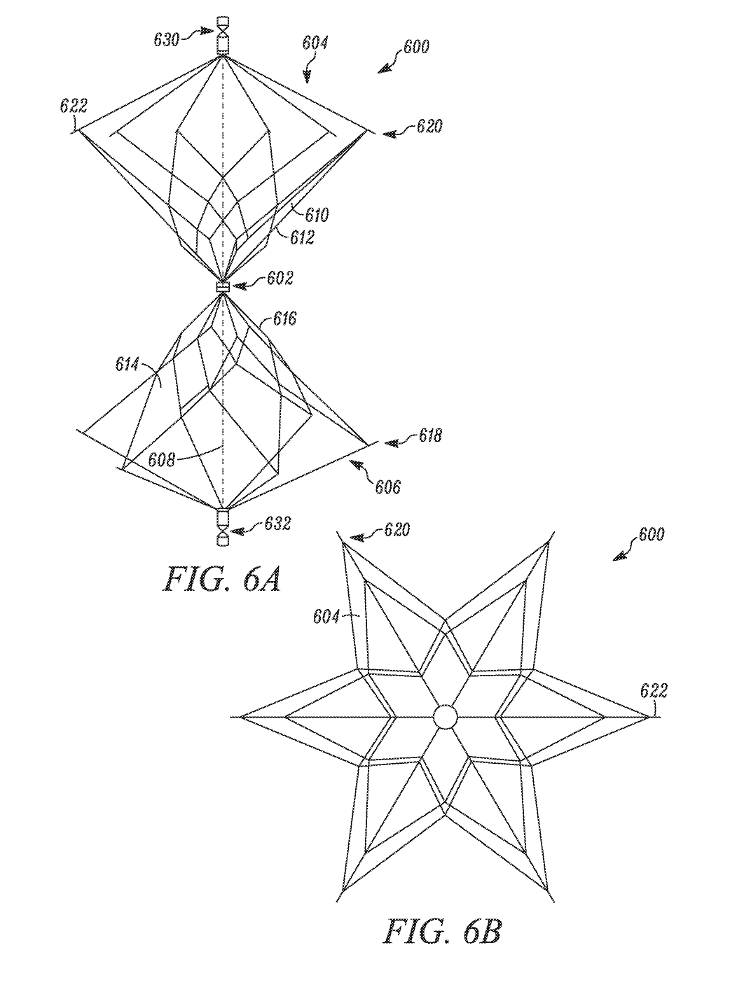

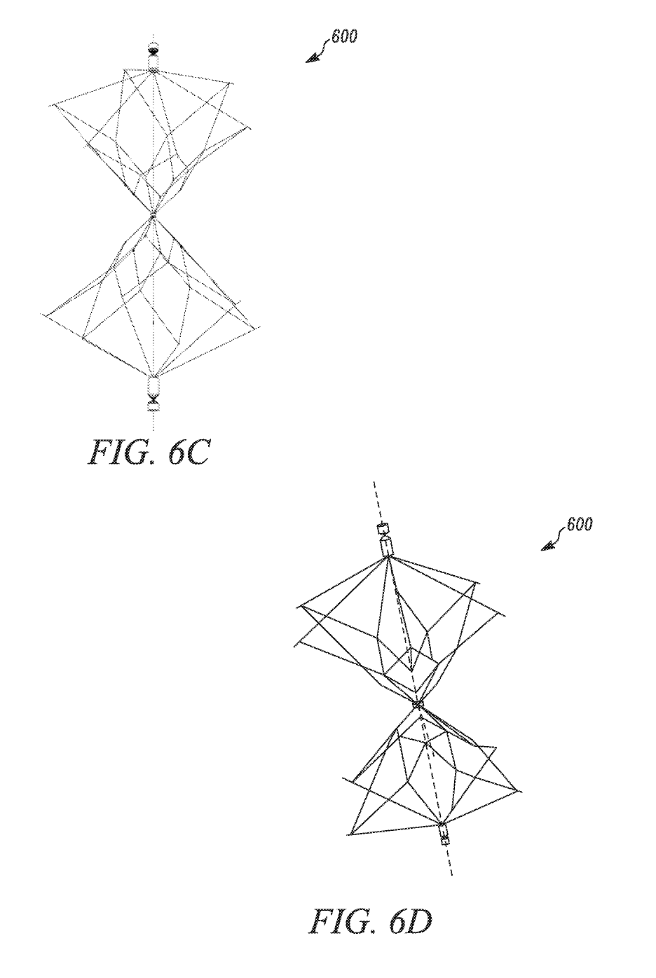

FIG. 6A shows a side view of an IVC filter 600. The filter 600 may include a center structure 602, a first basket formed by sides 610 and vertices 612, and a second basket 606 formed by sides 614 and vertices 616. The embodiment of FIG. 6A simplifies the polyhedral structure somewhat to use less structural elements. The filter 600 may also include retrieval fixtures 630, 632 to facilitate retrieval and one or more spikes 622 or the like. Additionally, the IVC filter 600 may include spikes 622 or the like to secure the filter 600 against movement when placed for use in the IVC. FIG. 6B shows a top view of the filter 600 of FIG. 6A. FIGS. 6C and 6D show perspective views of an IVC filter 600.

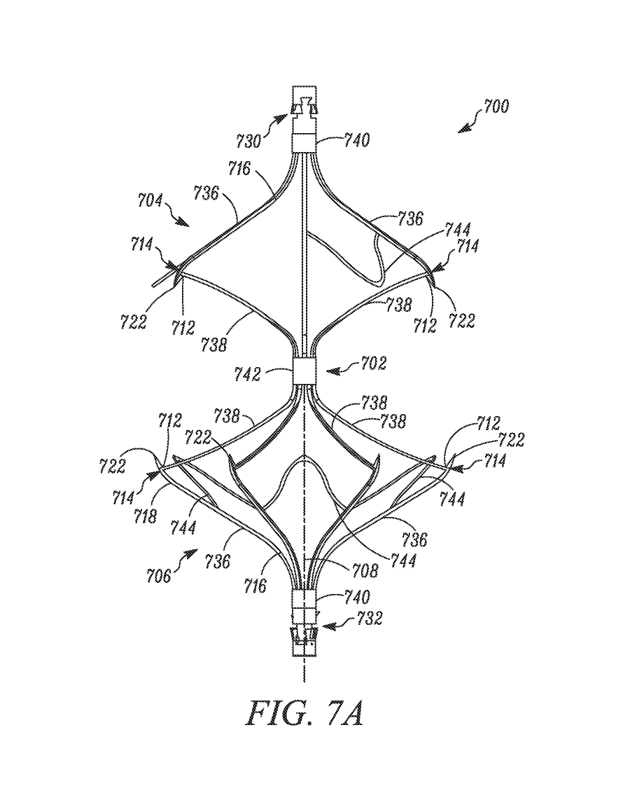

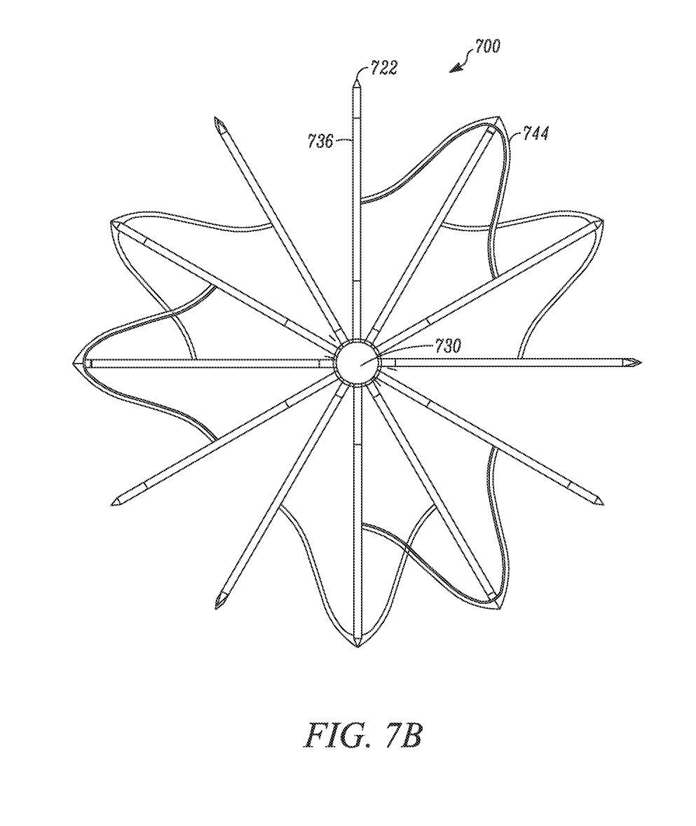

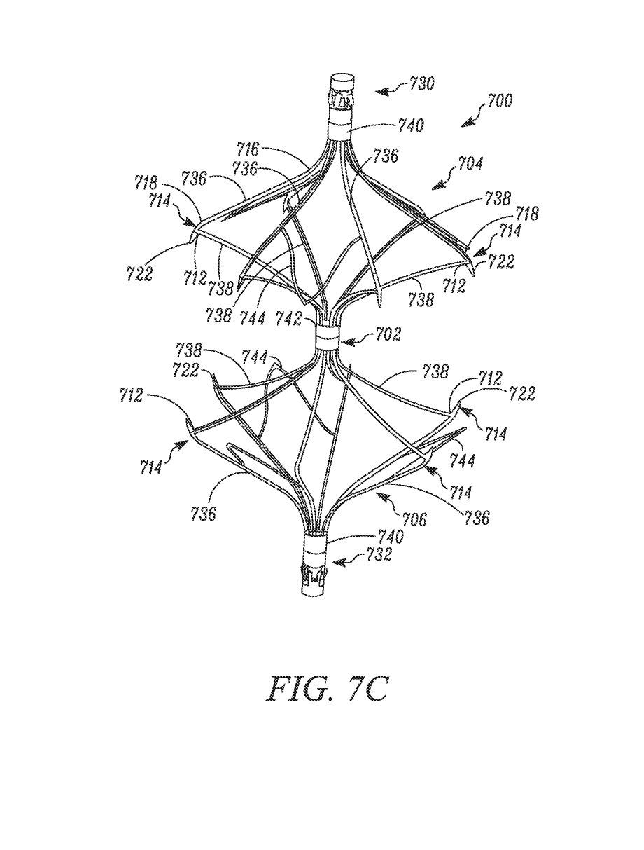

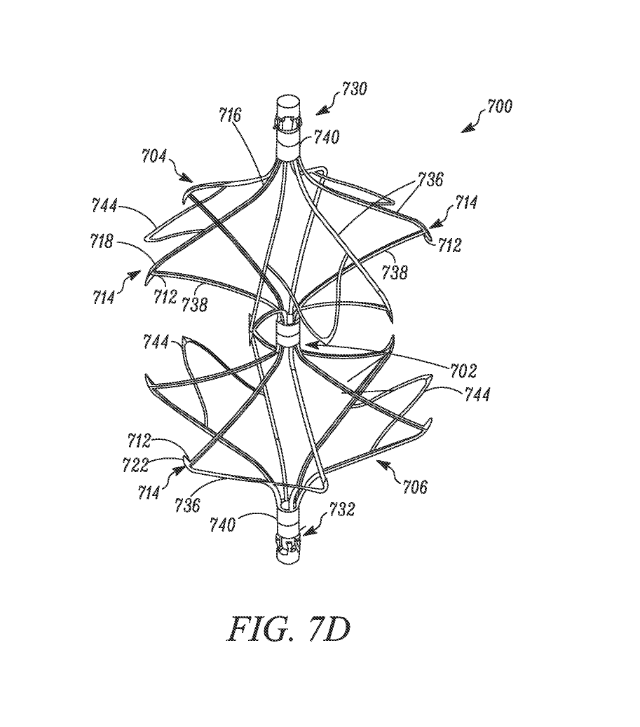

FIGS. 7A-7D show an IVC filter. The filter 700 may include a center structure 702, a first basket 704, and a second basket 706. The center structure 702 defines an axis 708 about which the center structure 702 may be constructed, and the center structure 702 creates a narrow section of the filter 700. The filter baskets 704, 706 may include outer legs 736 and inner legs 738, which may be slightly curved and connect at junctions 712 to form the closed shape of the filter baskets 704, 706. Because of the curved shape of the legs (in particular, the outer legs 136), the junctions 712 of the embodiment shown in FIGS. 7A-7D form substantially curved outer edges 714 disposed distally outward from the axis 708. Thus, the curved edges 714 are disposed distally outward from the center of the filter baskets 704, 706 at a plurality of locations, which may form the outermost edges of the baskets 704, 706 from the axis 708.

The outer legs 736 may connect to base structures 740 that engage with the retrieval fixtures 730, 732, while the inner legs 738 may connect to a central base structure 742 that engages with the center structure 702. In a filter, the base structure 740 may be integral with the retrieval fixture 730, 732. Also, in a filter, the central base structure 742 may be integral with the center structure 702. The filter 700 may also include support legs 744 that serve to stabilize the shape of the filter baskets 704, 706. The support legs 744 may also be used to increase the number of structural elements of the filter baskets 704, 706 for a number of desired reasons as will be known to those of skill in the art, e.g., increasing the filtering capability of the filter.

FIGS. 7A-7D depict three support legs 744 between the outer legs 736 of each filter basket 704, 706, but one of ordinary skill in the art will understand that more or less support legs 744 may be included in the filters described in the present application. The support legs 744 may be configured so that the basket 704, 706 is symmetric when viewed from the top and/or the bottom, or the support legs 744 may be configured so that the basket 704, 706 is asymmetric when viewed from the top and/or the bottom. Additionally, the support legs 744 may be located between the inner legs 738. In a filter, the support legs 744 may include a substantially v-shaped or u-shaped design. However, one skilled in the art will understand that the support legs 744 may include other shapes, including, but not limited to, a straight line, an s-shape, or a z-shape.

In the filter 700, one or more of the junctions 712 may include a wide angle between the outer legs 736 and the inner legs 738, which may be an angle greater than ninety degrees in order to mitigate possible piercing or rending of the cava wall. Additionally, the outer legs 736 of a filter 700 may project from the base structure 740 such that an angle of about thirty degrees is present between an outer leg and its neighboring outer leg. One skilled in the art will understand that this angle may be smaller or larger in embodiments of the filter 700. Further, in a filter 700, the inner legs 738 may project from the central base structure 742 such that an angle of about thirty degrees is present between an inner leg and its neighboring inner leg. One skilled in the art will understand that this angle may be smaller or larger in the IVC filters.

The curve of the outer legs 736 of a basket of an embodiment will now be described. The outer leg 736 may include a curve, or a plurality of curves as explained herein, e.g., a base curve 716 and an outer edge curve 718. The outer leg 736 may curve away from the base structure 740 forming a base curve 716 such that a focal point of the base curve 716 is disposed away from the axis 708 with respect to the basket 704, 706. The outer leg 736 may also curve toward the center axis 708 forming an outer edge curve 718 such that a focal point of the outer edge curve 718 is disposed toward the axis 708 with respect to the basket 704, 706. More generally, any variety and shape of curves may be used along the legs of the filter 700, provided they result in localized contact points for the IVC wall when the filter 700 is placed for use in order to mitigate attachment to or embedding in the IVC wall over time.

The curve of the inner legs 738 of a basket of an embodiment will now be described. The inner leg 738 may include a curve, or a plurality of curves. For example, the inner leg 738 may include a curve such that a focal point would be disposed toward an inner leg of an opposing basket.

The IVC filter 700 may include spikes 722 or the like. The spikes 722 may be disposed adjacent to, and/or protruding from, the curved outer edges 718.

The embodiment of FIGS. 7A-7D is designed to include less structural elements than other embodiments depicted in the figures. The embodiment of FIGS. 7A-7D may therefore include less filtration than other embodiments that are described herein. In other words, the IVC filter 700 may include less intersection points between structural members and thus, the geometry may provide for less filtration of a fluid flowing through the filter 700. One of ordinary skill in the art will understand that the filtering described herein may be adjusted based on the geometric shape of the filters, and the filter 700 of FIGS. 7A-7D may also be configured with more structural elements to increase filtration where desired.

The filter 700 in FIGS. 7A-7D is shown constructed out of thicker support structures than previous embodiments—it is not constructed from a fine material such as bulk wire. However, one skilled in the art will understand that the filter 700 and other embodiments shown/described herein (and otherwise included as embodiments) may be made from a fine material or otherwise and/or from a metal such as nitinol, stainless steel, or the like, or any materials suitable for small surgical filters.

The spikes that may be used to attach the filter to the IVC may be angled away from a direction normal to the IVC wall, which may prevent migration of each side (i.e., up-and-down migration), particularly where corresponding spikes on each filter basket are angled toward (or away from) each other. In this configuration, one set of spikes on one basket may be inclined, e.g., in the femoral direction while another set of spikes on the other basket may be inclined, e.g., in the jugular direction, thus concurrently resisting motion in either direction within the IVC and securing the filter 700 against axial drift along the vessel. The spikes may be shaped so that they do not interfere with retrieval, which is typically done through a tube, when the filter is collapsed.

The filters described herein may include spikes as shown in some of the figures. Additionally, the filters may include hooks, prongs, bosses, cams, gibs, fingers, legs, latches, wedges, posts, projections, spurs, or any other protrusions or the like suitable for attaching a filter to the IVC.

The filter may include a center structure that joins one of a first number of vertices of a filter basket to one of a second number of vertices.

The IVC filter may be shaped and sized to align at two points along an axis of the IVC corresponding to a greatest outside diameter of filter baskets included on the filter. The IVC filter may have a first end on a vertex of the first filter basket opposing the center structure and a second end on a vertex of the second filter basket opposing the center structure, where the IVC filter can be retrieved from a deployment within the IVC from the first end or the second end.

The IVC filter may include more than two filter baskets, e.g., the IVC filter may include one or more additional filter baskets joined to one another by one or more additional center structures to form a multi-basket IVC filter with three or more filter baskets. In other words, a filter may include three or more filter baskets.

The IVC filter may include a filter basket with a polyhedral structure having a vertex at a number of discrete locations. Further, an IVC filter may include a filter basket with at least one curved outer edge having a point distal from the axis of the IVC filter at a number of discrete locations.

A filter may include the capability for bilateral retrieval, which means that the filter may be retrieved from either end of the filter. The center structure of the filter may also include a means for decoupling during retrieval, such that each basket can be retrieved independently and/or retrieval may occur from both ends of the filter. Retrieval may be performed with a wire loop or the like as described herein. Therefore, to aid in retrieval of the filter, the retrieval fixture may include a latch or the like, which catches the wire loop or the like upon retrieval. Additionally, the spikes of the filter may be shaped to catch a wire loop or the like to aid in retrieval.

A filter may be constructed out of one material or a plurality of materials. Yet another filter may be constructed such that it can be made from one piece of material (e.g., one length of wire bent and/or cut into shape, one length of tube bent and/or cut into shape, or the like). The filters may also be laser cut or the like.

The center structure 102 in a filter may include a hollow structure (e.g., a hollow tube or the like) or it may be a solid or substantially solid piece or set of pieces.

The shape of the structural elements of the filters, e.g., the legs 736, 738 in IVC filter 700, may be in the form of tubes, wires, or the like, or may be in the form of elongated structures with any substantially polygon cross-section or the like (e.g., a polygon with rounded edges).

A filter may include baskets that may have a substantially polyhedron shape that can expand and/or contract. The substantially polyhedron shape may include substantially straight edges/sides and/or substantially rounded/curved edges/sides. The vertices and sides of the polyhedron shapes of the baskets described above afford greater structural integrity, and can resiliently retain the polyhedral shape against the forces applied by the IVC walls. As another advantage, this resilient shape helps to ensure that spikes or other anchoring mechanisms securely affix to the vessel walls.

As another advantage, the polyhedral and other baskets with small, localized contact points can reduce the contact area of the filters against the IVC wall when place for use by providing discrete locations (e.g., vertices of the polyhedral, or curved extremities of curving edges in FIGS. 7A-7D) where the filter meets the IVC wall when placed for use. This significantly improves over certain prior art filters that use long, straight or curved edges that rest along their length against the IVC wall when deployed, posing increased risks of attachment to or embedding in the IVC wall with prolonged use.

As another advantage, the substantially symmetric nature of the filters described herein facilitates bidirectional placement, use, and retrieval. Certain asymmetric filters described above provide similar advantages through the use of a center section having a smaller cross section (along the axis) than the cross section of the filter baskets so that the axis of the filter naturally aligns to the IVC when deployed.

The embodiments include the IVC filter described herein, as well as the methods of making and methods of using the IVC filter described herein.

While particular embodiments of the invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. An inferior vena cava (IVC) filter formed of a number of sides joined at a number of vertices in a deployed form comprising:
a center structure having an axis;
a first filter basket including a first number of sides each coupled at a first number of vertices to form a first polyhedron, a first one of the first number of vertices coupled to the center structure and three or more of the vertices positioned about the axis; and
a second filter basket including a second number of sides each coupled at a second number of vertices to form a second polyhedron, a first one of the second number of vertices coupled to the center structure and three or more of the vertices positioned about the axis;
wherein none of the sides on a convex hull of the device are parallel to the axis.

2. The IVC filter of claim 1 wherein none of the sides of the filter baskets lie on a perimeter of an axial cross section of the device.

3. The IVC filter of claim 1 wherein the center structure joins one of the first number of vertices to one of the second number of vertices.

4. The IVC filter of claim 1 wherein the first filter basket and the second filter basket are formed of surgical stainless steel.

5. The IVC filter of claim 1 wherein the sides of the first filter basket and the second filter basket are formed of a number of lengths of stainless steel wire with no free ends.

6. The IVC filter of claim 5 wherein none of the number of lengths terminate at the center structure.

7. The IVC filter of claim 5 wherein all of the number of lengths terminate at a first retrieval fixture coupled to the first filter basket or a second retrieval fixture coupled to the second filter basket.

8. The IVC filter of claim 1 wherein the first filter basket and the second filter basket are formed of a shape memory alloy.

9. The IVC filter of claim 8 wherein the shape memory alloy includes nickel titanium.

10. The IVC filter of claim 1 wherein the first filter basket and the second filter basket are formed of an MRI-compatible material.

11. The IVC filter of claim 1, wherein the IVC filter is shaped and sized to align at two points along an axis of the IVC corresponding to a greatest outside diameter of the first filter basket and a greatest outside diameter of the second filter basket.

12. The IVC filter of claim 1 wherein the IVC filter has a first end on a vertex of the first filter basket opposing the center structure and a second end on a vertex of the second filter basket opposing the center structure, wherein the IVC filter can be retrieved from a deployment within the IVC from the first end or the second end.

13. The IVC filter of claim 1 wherein the first filter basket and the second filter basket form two sequential filters in a path of flow through the IVC.

14. The IVC filter of claim 1 wherein the first filter basket and the second filter basket are collapsible from the deployed form into a collapsed form to permit deployment and retrieval through a minimally invasive surgical tool.

15. The IVC filter of claim 1 wherein the first filter basket and the second filter basket are collapsible from the deployed form to a collapsed form to permit deployment and retrieval using an endovascular technique.

16. The IVC filter of claim 15 wherein the IVC filter elastically returns to the deployed form from the collapsed form in the absence of external forces.

17. The IVC filter of claim 15 wherein the IVC filter is formed of a shape memory alloy that returns to the deployed form upon heating above a transition temperature.

18. A filter comprising:
a first filter basket having a first polyhedral geometry and no free ends, the first polyhedral geometry including a first number of contact points at a number of vertices arranged to contact an interior wall of an inferior vena cava (IVC) when the filter is placed for use in the IVC;
a second filter basket having a second polyhedral geometry and no free ends, the second polyhedral geometry including a second number of contact points at a number of vertices arranged to contact the interior wall of when the IVC filter is placed for use in the IVC; and
a narrow center joining the first filter basket and the second filter basket into a self-centering, bi-directional, IVC filtering structure.

19. The filter of claim 18 wherein one or more of the contact points has a spike to secure the filter to the interior wall of the IVC.

20. The filter of claim 19 wherein the spike is about two to three millimeters long.

21. The filter of claim 18 wherein the first filter basket and the second filter basket are collapsible to facilitate deployment and retrieval through a minimally invasive endovascular tool.

22. The filter of claim 18 wherein one or more of the contact points is formed by a vertex having a wide angle.

23. The filter of claim 22 wherein the wide angle is greater than ninety degrees.

24. The filter of claim 22 wherein all of the contact points are formed by vertices having a wide angle.

25. The filter of claim 22 wherein the one or more of the contact points includes a spike to secure the filter to the interior wall of the IVC.

26. The filter of claim 18 further comprising a first retrieval fixture on the first filter basket, the first retrieval fixture including a cylinder with an indentation configured to removably and replaceably receive a snare.

27. The filter of claim 26 further comprising a second retrieval fixture on the second filter basket.

28. The filter of claim 27 wherein the first filter basket and the second filter basket are formed by a number of wires, and wherein the first retrieval fixture and the second retrieval fixture secure a number of terminal ends of the number of wires.

29. The filter of claim 18 further comprising a retrieval fixture on the first filter basket, the retrieval fixture including a cylinder with a narrowed sidewall shaped to removably and replaceably receive a snare.

30. The filter of claim 18 wherein no sides of the first filter basket and the second filter basket contact the interior wall of the IVC when the filter is placed for use in the IVC.

31. The filter of claim 18 wherein no sides of the first filter basket and the second filter basket are in parallel contact with the interior wall of the IVC when the filter is placed for use in the IVC.

32. The filter of claim 18 wherein the first filter basket forms a filtering structure to catch clots within blood flowing through the IVC when the filter is placed for use.

33. The filter of claim 32 wherein the second filter basket forms a second filtering structure to catch clots within blood flowing through the IVC when the filter is placed for use.

34. An inferior vena cava (IVC) filter comprising:
a first filter basket having at least one cross section shaped and sized to fit within and engage a wall of an inferior vena cava at a first number of discrete locations;
a second filter basket having at least one cross section shaped and sized to fit within and engage the wall of the inferior vena cava at a second number of discrete locations; and
a center structure joining the first filter basket and the second filter basket, the center structure having a cross section with a diameter smaller than the cross section of the first filter basket and the cross section of the second filter basket.

35. The IVC filter of claim 34 further comprising one or more additional filter baskets joined to one another by one or more additional center structures to form a multi-basket IVC filter with three or more filter baskets.

36. The IVC filter of claim 34 wherein the first filter basket includes a polyhedral structure having a vertex at each of the first number of discrete locations.

37. The IVC filter of claim 34 wherein the first filter basket includes at least one curved outer edge having a point distal from an axis of the IVC filter at one of the first number of discrete locations.

1-17. (canceled)

18. A filter comprising:
a first filter basket having a first polyhedral geometry and no free ends, the first polyhedral geometry including a first number of contact points at a number of vertices arranged to contact an interior wall of an inferior vena cava (IVC) when the filter is placed for use in the IVC;
a second filter basket having a second polyhedral geometry and no free ends, the second polyhedral geometry including a second number of contact points at a number of vertices arranged to contact the interior wall of when the IVC filter is placed for use in the IVC; and
a narrow center joining the first filter basket and the second filter basket into a self-centering, bi-directional, IVC filtering structure.

19. The filter of claim 18 wherein one or more of the contact points has a spike to secure the filter to the interior wall of the IVC.

20. The filter of claim 19 wherein the spike is about two to three millimeters long.

21. The filter of claim 18 wherein the first filter basket and the second filter basket are collapsible to facilitate deployment and retrieval through a minimally invasive endovascular tool.

22. The filter of claim 18 wherein one or more of the contact points is formed by a vertex having a wide angle.

23. The filter of claim 22 wherein the wide angle is greater than ninety degrees.

24. The filter of claim 22 wherein all of the contact points are formed by vertices having a wide angle.

25. The filter of claim 22 wherein the one or more of the contact points includes a spike to secure the filter to the interior wall of the IVC.

26. The filter of claim 18 further comprising a first retrieval fixture on the first filter basket, the first retrieval fixture including a cylinder with an indentation configured to removably and replaceably receive a snare.

27. The filter of claim 26 further comprising a second retrieval fixture on the second filter basket.

28. The filter of claim 27 wherein the first filter basket and the second filter basket are formed by a number of wires, and wherein the first retrieval fixture and the second retrieval fixture secure a number of terminal ends of the number of wires.

29. The filter of claim 18 further comprising a retrieval fixture on the first filter basket, the retrieval fixture including a cylinder with a narrowed sidewall shaped to removably and replaceably receive a snare.

30. The filter of claim 18 wherein no sides of the first filter basket and the second filter basket contact the interior wall of the IVC when the filter is placed for use in the IVC.

31. The filter of claim 18 wherein no sides of the first filter basket and the second filter basket are in parallel contact with the interior wall of the IVC when the filter is placed for use in the IVC.

32. The filter of claim 18 wherein the first filter basket forms a filtering structure to catch clots within blood flowing through the IVC when the filter is placed for use.

33. The filter of claim 32 wherein the second filter basket forms a second filtering structure to catch clots within blood flowing through the IVC when the filter is placed for use.

34. An inferior vena cava (IVC) filter comprising:
a first filter basket having at least one cross section shaped and sized to fit within and engage a wall of an inferior vena cava at a first number of discrete locations;
a second filter basket having at least one cross section shaped and sized to fit within and engage the wall of the inferior vena cava at a second number of discrete locations; and
a center structure joining the first filter basket and the second filter basket, the center structure having a cross section with a diameter smaller than the cross section of the first filter basket and the cross section of the second filter basket.

35. The IVC filter of claim 34 further comprising one or more additional filter baskets joined to one another by one or more additional center structures to form a multi-basket IVC filter with three or more filter baskets.

36. The IVC filter of claim 34 wherein the first filter basket includes a polyhedral structure having a vertex at each of the first number of discrete locations.

37. The IVC filter of claim 34 wherein the first filter basket includes at least one curved outer edge having a point distal from an axis of the IVC filter at one of the first number of discrete locations.
